(19) United States
(12) Patent Application Publication
(10) Pub. No.: US 2025/0182272 A1
(43) Pub. Date: Jun. 5, 2025

(54) MEDICAL IMAGE AUXILIARY DETECTION METHOD USING CBAM MECHANISM-BASED RESIDUAL NETWORK

(71) Applicant: CHONGQING UNIVERSITY OF ARTS AND SCIENCES, Chongqing (CN)

(72) Inventors: Xinqiang MA, Chongqing (CN); Yi HUANG, Chongqing (CN); Kang LI, Chongqing (CN); Zhigang YANG, Chongqing (CN); Daizhong LUO, Chongqing (CN); Wang LI, Chongqing (CN); Qiang LI, Chongqing (CN); Yang KANG, Chongqing (CN); Zhongjie WAN, Chongqing (CN); Xingyan YAO, Chongqing (CN); Antong ZHOU, Chongqing (CN); Youyuan LIU, Chongqing (CN); Songtao JIANG, Chongqing (CN)

(21) Appl. No.: 18/840,788

(22) PCT Filed: Dec. 15, 2022
(86) PCT No.: PCT/CN2022/139162

§ 371 Date: Aug. 22, 2024

(30) Foreign Application Priority Data
Jul. 22, 2022 (CN) 202210868339.8

(51) Int. Cl. G06T 7/00; A61B 6/00; A61B 6/50; G06T 3/40; G06T 5/20; G06T 5/60; G06T 5/70; G06T 7/11; G06T 11/00; G16H 50/20

(52) U.S. Cl. CPC G06T 7/0012; G06T 3/40; G06T 5/20; G06T 5/60; G06T 5/70; G06T 7/11; G06T 11/00; G16H 50/20; A61B 6/50; A61B 6/5217; G06T 2207/10081; G06T 2207/10116; G06T 2207/20081; G06T 2207/20084; G06T 2207/30061; G06T 2207/30096; G06T 2210/41; G06T 2210/62

(57) ABSTRACT

Provided is a medical image auxiliary detection method using a Convolutional Block Attention Module (CBAM) mechanism-based residual network. The method includes the following steps: S1: acquiring a medical image (which is a lung cxr medical image), and clipping and normalizing the medical image; S2: performing data transformation on the normalized medical image; S3: establishing a network model on the basis of convolutional autoencoding, a feature extraction method using a spatial-and-channel attention mechanism, and a Hierarchical-Split (HS)-block module; and S4: inputting the medical image obtained after the data transformation into the network model for prediction, and visualizing a predicted lesion region. By introducing a CBAM mechanism and an HS-block residual structure, the method enhances the lung X-ray feature extraction capability of the model, and improves the detection accuracy; the method is used for assisting traditional manual screening of lung X-ray images, and can improve the detection efficiency.

### CROSS REFERENCE TO RELATED APPLICATION

The present disclosure is a national stage application of International Patent Application No. PCT/CN2022/139162, filed on Dec. 25, 2022, which claims the benefit and priority of Chinese Patent Application No. 202210868339.8, filed with the China National Intellectual Property Administration (CNIPA) on Jul. 22, 2022, and entitled “MEDICAL IMAGE AUXILIARY DETECTION METHOD USING CBAM MECHANISM-BASED RESIDUAL NETWORK”, which is incorporated herein by reference in its entirety.

### TECHNICAL FIELD

The present disclosure relates to the technical field of medical image detection, and in particular, to a medical image auxiliary detection method using a Convolutional Block Attention Module (CBAM) mechanism-based residual network.

### BACKGROUND

The key to controlling the epidemic is early detection, early isolation, and early treatment. It is crucial to assist doctors in quickly identifying COVID-19 patients. Currently, the main testing methods include nucleic acid testing, antigen testing, and antibody testing. Using medical images for detection has advantages such as convenience, high sensitivity, and repeatability. Chest medical imaging for COVID-19 diagnosis includes two major technologies: chest X-rays and computed tomography (CT) scan images. These imaging technologies provide important evidence for doctors in making diagnoses. Both chest X-rays and CT scan images of the lungs play crucial roles in early screening and diagnosis of lesions. However, the large number of patients and the rapid evolution of the disease pose a significant challenge to radiologists due to the substantial amount of images generated during follow-up examinations. Especially in severely affected areas, rapidly screening and diagnosing a large number of suspected COVID-19 patients present a huge challenge to radiologists. In recent years, numerous studies have focused on automatic identification and assisted diagnosis based on medical images. The recognition of medical images has become a hot topic and entry for deep learning extending from the field of computer science to medicine. The recognition and detection of medical images based on deep learning not only alleviates the strain on medical resources but also helps avoid errors and missed diagnoses caused by human factors. Particularly during disease outbreaks, using computers to assist doctors in making diagnoses based on medical images significantly improves diagnostic efficiency and reduces the risk of infection for healthcare workers and the general public. Therefore, introducing artificial intelligence for assisting in the detection of medical images offers benefits such as facilitating patient treatment, alleviating pressure on medical resources, and enhancing detection accuracy.

In conclusion, there have been related research reports on using chest X-ray imaging for COVID-19 detection. However, in the medical diagnostic environment, the large amount of data and the highly contagious nature of COVID-19 impose stricter requirements on identification speed and accuracy. For large-scale medical lung imaging systems, more efficient and precise image classification and visualization methods are still lacking.

### SUMMARY

Accordingly, it is necessary to provide a medical image auxiliary detection method using a CBAM mechanism-based residual network, to address the issue of low efficiency in medical image diagnosis.

To achieve the above objective, the present disclosure provides the following technical solutions:

A medical image auxiliary detection method using a CBAM mechanism-based residual network, including the following steps:
S1: acquiring a medical image, and clipping and normalizing the medical image;
S2: performing data transformation on the normalized medical image;
S3: establishing a network model on the basis of convolutional autoencoding, a feature extraction method using a spatial-and-channel attention mechanism, and a Hierarchical-Split (HS)-block module; and
S4: inputting the medical image obtained after the data transformation into the network model for prediction, and visualizing a predicted lesion region.

Preferably, the medical image is a lung chest X-ray (cxr) medical image.

Preferably, step S1 specifically includes the following steps:
S11: directly scaling the medical image to an image with a size suitable for input to the network model (224px, 224px);
S12: converting the image into a grayscale image through channel reduction using the following formula: GRAY=B*0.114+G*0.387+R*0.299, and reducing parameters during model training, where B represents a blue component, G represents a green component, and R represents a red component in a three-channel image;
S13: converting the grayscale image into a tensor form (B, C, H, W), where B represents a batch size, C represents the number of image channels, H represents an image height, and W represents an image width; and
S14: normalizing the image obtained in S13 using a Normalize function to facilitate model convergence.

Preferably, step S2 specifically includes the following steps:
S21: performing data augmentation through central rotation of the normalized medical image to increase the amount of training data; and
S22: removing Gaussian noise from the normalized medical image using Gaussian filtering, where resulting data is used as input for subsequent training.

Preferably, step S3 specifically includes the following steps:
S31: constructing a Res2Net residual network structure based on a ResNet network architecture, where the ResNet structure includes 34 convolutional layers, two pooling layers, and one fully connected layer, replacing original 3×3 convolutions with residual groups on different channels;
S32: constructing a CBAM attention mechanism by combining a channel attention mechanism and a spatial attention mechanism, and inserting the constructed CBAM attention mechanism into the Res2Net residual network structure; and
S33: constructing an HS-block multi-level separable module and adding the HS-block multi-level separable module to a head of an entire network, allowing the network to learn stronger feature information without increasing computational complexity.

Preferably, a process of the channel attention mechanism is described as follows:

Global average pooling (AvgPool) and global maximum pooling (MaxPool) are performed on a width and a height of a network feature map; channel attention weights are obtained through a multi-layer perceptron (MLP); the obtained weights are summed element-wise; finally, the weights are normalized using a Sigmoid function, and are then multiplied channel-wise to the original feature map, with a formula as follows:

$$\begin{matrix}{M}_{c} ( F ) = σ ( MLP ( AvgPool ( F ) ) + MLP ( MaxPool ( F ) ) ) \\ = σ ( {W}_{1} ( {W}_{0} ( {F}_{avg}^{c} ) ) + {W}_{1} ( {W}_{0} ( {F}_{max}^{c} ) ) )\end{matrix}$$

where F represents an input weighted feature map, W₀, W₁ represents a fully connected layer, σ represents a sigmoid method, F_{avg}^{c} represents a feature after global average pooling, Fₘₐₓ^{c} represents a feature after global maximum pooling, and an operation result of the channel attention mechanism serves as input for the spatial attention mechanism.

Preferably, a process of the spatial attention mechanism is described as follows:

With input from the channel attention mechanism, global maximum pooling (MaxPool) and global average pooling (AvgPool) are performed on the feature map based on channels; then, the dimensionality is reduced to 1D through convolution operations, and attention features are generated through a Sigmoid function, with a formula as follows:

$${M}_{s} ( F ) = σ ( {\int }^{7 \times 7} ( [ AvgPool ( F ) ; MaxPool ( F ) ] ) ) = σ ( {\int }^{7 \times 7} ( [ {F}_{avg}^{s} ; {F}_{max}^{s} ] ) )$$

where F represents an input weighted feature map, and σ represents a sigmoid function.

Preferably, said inserting the constructed CBAM attention mechanism into the Res2Net residual network structure specifically includes:

inserting the constructed CBAM attention mechanism into the last layer of each residual block of ResNet.

Preferably, said constructing the HS-block multi-level separable module specifically includes:

dividing the feature map into groups by channels, and performing cross-combination and convolution on different groups, which facilitates the extraction of abstract information.

Preferably, step S4 specifically includes:

extracting features from the model based on a Grad-CAM++ algorithm, plotting a heatmap, and overlaying the heatmap on an original image with 0.3 opacity.

Preferably, the Grad-CAM++ algorithm is specifically as follows:

A score for a specific class in a feature map is derived from a dot product of weights and the feature map, with a formula as follows: Y^{c}=Σₖwₖ^{c}·ΣᵢΣⱼA_{i,j}^{k}, where c represents a class, (i, j) represents a position of a feature value in the feature map, k represents a channel, Y represents a contribution to the class c, and A represents the feature map; a corresponding heatmap formula is as follows: L_{i,j}^{c}=Σₖwₖ^{c}·A_{i,j}^{k}, where A_{i,j}^{c} represents a value at the position (i, j) in the feature map, Wₖ^{c} represents a fully connected weight for the class c regarding the channel k, and L_{i,j}^{c} represents a contribution of the position (i, j) in the feature map to the class c; the calculation of the weights uses gradients and a ReLU activation function for improvement, with a formula as follows:

$${w}_{k}^{c} = {\sum }_{i} {\sum }_{j} {∝}_{ij}^{kc} \cdot relu ( \frac{\partial {Y}^{c}}{\partial {A}_{ij}^{k}} ) ,$$

where ∝_{i,j}^{kc} represents a weighted coefficient of a pixel gradient for the class c and A^{k} in the feature map, relu( ) represents a ReLU activation function, A^{k} represents a value at the position (i, j) in the feature map, and Y^{c} represents a differentiable function used for activation of A^{k}.

Compared with the prior art, the present disclosure achieves following beneficial effects:

The present disclosure provides an auxiliary detection method for COVID-19 lung images using a CBAM mechanism-based residual network. It achieves a high-precision COVID-19 X-ray assisted diagnostic algorithm, optimizes traditional manual case screening solutions, and integrates the attention mechanism with the HS-block module to enhance inference accuracy, thereby meeting the demands for recognizing a large number of images in medical diagnosis.

### BRIEF DESCRIPTION OF THE DRAWINGS

To describe embodiments of the present disclosure or technical solutions in the prior art more clearly, the accompanying drawings required in the embodiments are briefly introduced below. Obviously, the accompanying drawings described below are only some embodiments of the present disclosure. Those of ordinary skill in the art can also obtain other accompanying drawings according to these accompanying drawings without creative efforts.

FIG. 1 is a schematic flowchart of a medical image auxiliary detection method using a CBAM mechanism-based residual network according to the present disclosure;

FIGS. 2A-2D illustrate an effect after image transformation according to the present disclosure;

FIG. 3 illustrates an overall framework of a network model according to the present disclosure;

FIG. 4 is a framework diagram of an attention mechanism according to the present disclosure;

FIG. 5 is a framework diagram of an HS-block involved in the present disclosure;

FIG. 6 shows changes of acc and loss during case training according to the present disclosure;

FIGS. 7A-7C illustrate a case testing effect according to the present disclosure, including an ROC curve, a PR curve, and a confusion matrix; and

FIG. 8 illustrates a specific implementation effect of the present disclosure.

### DETAILED DESCRIPTION OF THE EMBODIMENTS

The technical solutions of the embodiments of the present disclosure are clearly and completely described below with reference to the drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

To make the above objectives, features, and advantages of the present disclosure clearer and more comprehensible, the present disclosure will be further described in detail below with reference to the accompanying drawings and the specific embodiments.

Data used in the present disclosure comes from eight datasets of three open source websites: Kaggle, RSNA, and Github, as shown in the table below:

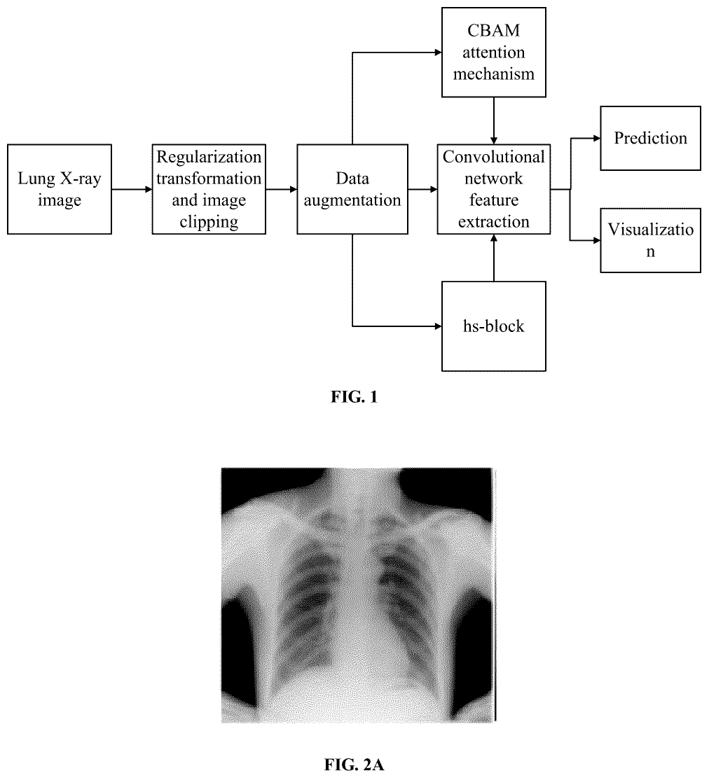

TABLE 1

| Dataset | Amount of data |
|---|---|
| Actualmed-COVID-chestxray-dataset | covid-19: 58; normal: 127 |
| COVID_IEEE | covid: 536; normal: 668 |
| COVID-19 Detection X-Ray Dataset | covid: 129; normal: 2231 |
| COVID-19 Radiography Database | covid: 3616; normal: 10192 |
| Covid19-dataset | covid: 137; normal: 90 |
| covid-chestxray-dataset | covid: 342; normal: 15 |
| FIG. 1-COVID-chestxray-dataset | covid: 35; normal: 3 |
| RSNA Pneumonia Detection Challenge | normal: 8851 |

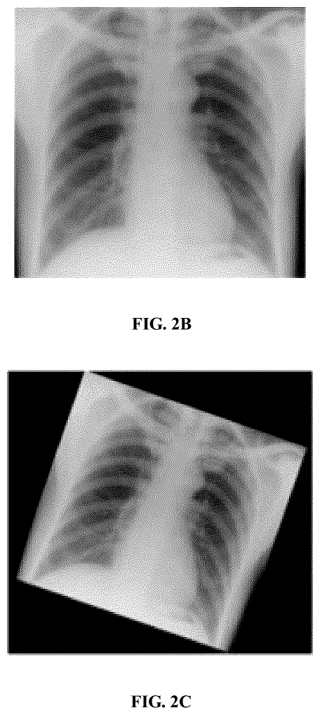

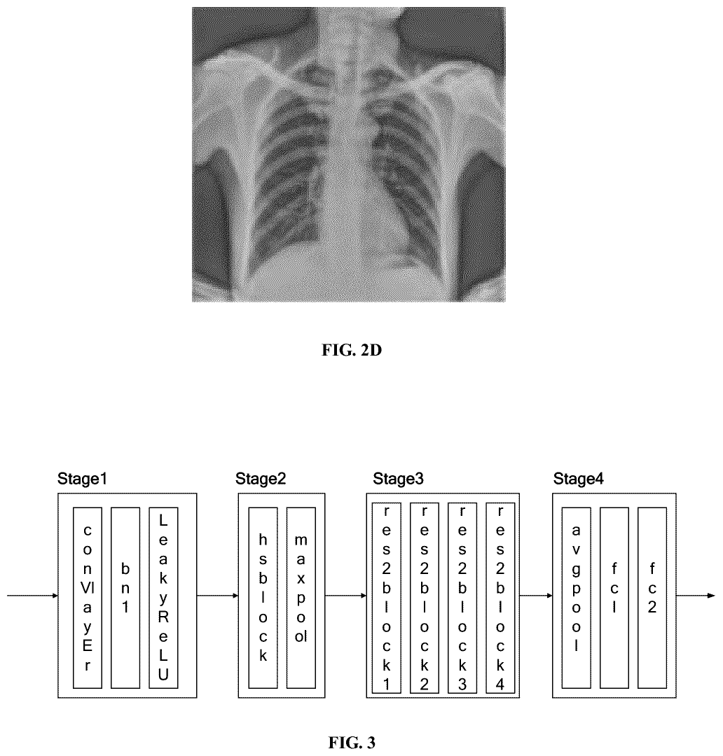

The present disclosure provides a medical image auxiliary detection method using a CBAM mechanism-based residual network. As shown in FIG. 1, the method includes the following steps:
S1: Acquire a medical image, and clip and normalize the medical image, where the medical image is a lung cxr medical image.
S2: Perform data transformation on the normalized medical image, with the effect shown in FIGS. 2A-2D.
S3: Establish a network model on the basis of convolutional autoencoding, a feature extraction method using a spatial-and-channel attention mechanism, and an HS-block module, where the framework of the network model is as shown in FIG. 3.
S4: Input the medical image obtained after the data transformation into the network model for prediction, and visualize a predicted lesion region.

The present disclosure uses a computer device to execute the foregoing steps. The computer device includes a memory and a processor, where the memory stores a computer program, and the computer program is executed by the processor to perform the steps of the medical image auxiliary detection method using a CBAM mechanism-based residual network.

Each step is described in detail below.

Specifically, step S1 specifically includes the following steps:
S11: Directly scale the medical image to an image with a size suitable for input to the network model (224px, 224px).
S12: Convert the image into a grayscale image through channel reduction using the following formula: GRAY=B*0.114+G*0.387+R*0.299, and reduce parameters during model training, where B represents a blue component, G represents a green component, and R represents a red component in a three-channel image.
S13: Convert the grayscale image into a tensor form (B, C, H, W), where B represents a batch size, C represents the number of image channels, H represents an image height, and W represents an image width.
S14: Normalize the image obtained in S13 using a Normalize function to facilitate model convergence.

Specifically, step S2 specifically includes the following steps:
S21: Perform data augmentation through central rotation of the image to increase the amount of training data.
S22: Remove Gaussian noise from the image using Gaussian filtering, where resulting data is used as input for subsequent training.

Specifically, step S3 specifically includes the following steps:
S31: Construct a Res2Net residual network structure based on a ResNet network architecture, where the ResNet structure includes 34 convolutional layers, two pooling layers, and one fully connected layer, replacing original 3×3 convolutions with residual groups on different channels.
S32: Construct a CBAM attention mechanism by combining a channel attention mechanism and a spatial attention mechanism, and insert the constructed CBAM attention mechanism into the Res2Net residual network structure.
S33: Construct an HS-block multi-level separable module and add the HS-block multi-level separable module to the head of an entire network, allowing the network to learn stronger feature information without increasing computational complexity.

Specifically, a process of the channel attention mechanism is described as follows:

Global average pooling (AvgPool) and global maximum pooling (MaxPool) are performed on a width and a height of a network feature map; channel attention weights are obtained through a multi-layer perceptron (MLP); the obtained weights are summed element-wise; finally, the weights are normalized using a Sigmoid function, and are then multiplied channel-wise to the original feature map, with a formula as follows:

$$\begin{matrix}{M}_{c} ( F ) = σ ( MLP ( AvgPool ( F ) ) + MLP ( MaxPool ( F ) ) ) \\ = σ ( {W}_{1} ( {W}_{0} ( {F}_{avg}^{c} ) ) + {W}_{1} ( {W}_{0} ( {F}_{max}^{c} ) ) )\end{matrix}$$

where F represents an input weighted feature map, W₀, W₁ represents a fully connected layer, σ represents a sigmoid method, and an operation result of the channel attention mechanism serves as input for the spatial attention mechanism.

A process of the spatial attention mechanism is described as follows:

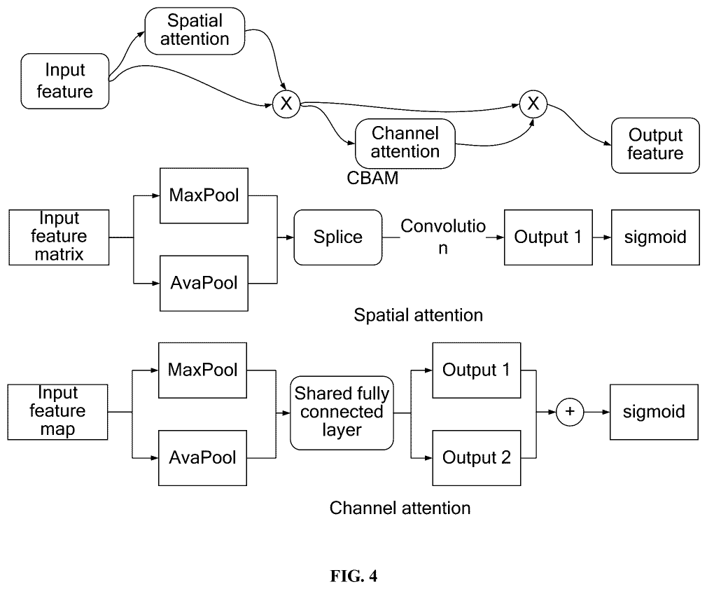

With input from the channel attention mechanism, global maximum pooling (MaxPool) and global average pooling (AvgPool) are performed on the feature map based on channels; then, the dimensionality is reduced to 1D through convolution operations, and attention features are generated through a Sigmoid function (the block diagram is as shown in FIG. 4), with a formula as follows:

$${M}_{s} ( F ) = σ ( {\int }^{7 \times 7} ( [ AvgPool ( F ) ; MaxPool ( F ) ] ) ) = σ ( {\int }^{7 \times 7} ( [ {F}_{avg}^{s} ; {F}_{max}^{s} ] ) )$$

where F represents an input weighted feature map, and σ represents a sigmoid function.

The step of constructing the HS-block multi-level separable module specifically includes:

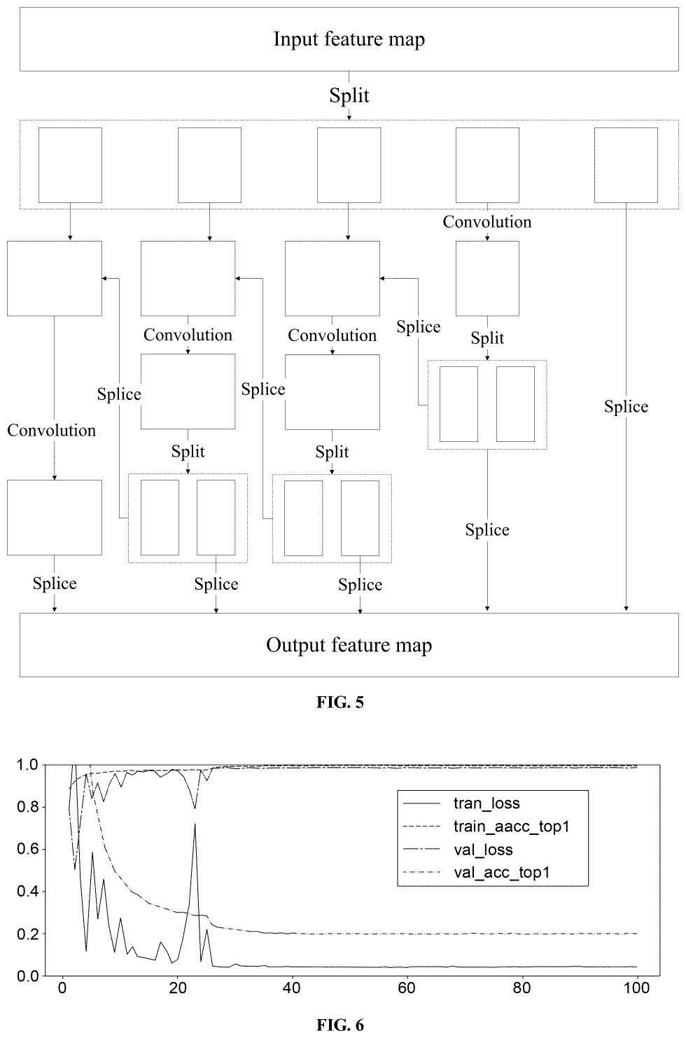

dividing the feature map into groups by channels, and performing cross-combination and convolution on different groups, which facilitates the extraction of abstract information, where the corresponding structure is as shown in FIG. 5.

Specifically, step S4 specifically includes:
extracting features from the model based on a Grad-CAM++ algorithm, plotting a heatmap, and overlaying the heatmap on an original image with 0.3 opacity.

The Grad-CAM++ algorithm is specifically as follows:

A score for a specific class in a feature map is derived from a dot product of weights and the feature map, with a formula as follows: Y^{c}=Σₖwₖ^{c}·ΣᵢΣⱼA_{i,j}^{k}a corresponding heatmap formula is as follows: L_{i,j}^{c}=Σₖwₖ^{c}·A_{i,j}^{k}, where A_{i,j}^{c} calculation of weights uses gradients and a ReLU activation function for improvement, with a formula as follows:

$${w}_{k}^{c} = {\sum }_{i} {\sum }_{j} {∝}_{ij}^{kc} \cdot relu ( \frac{\partial {Y}^{c}}{\partial {A}_{ij}^{k}} ) .$$

The present disclosure describes detection of the current model using accuracy (Acc), recall, balanced F-score (F1 Score), sensitivity, specificity, and AUC. Accuracy indicates the correctness

$$Acc = \frac{( TP + TN )}{( TP + TN + FP + FN )} ,$$

of predictions, where TP represents that a positive sample is predicted as a positive sample, TN represents that a negative sample is predicted as a negative sample, FP represents that a negative sample is predicted as a positive sample, and FN represents that a positive sample is predicted as a negative sample. Specificity indicates a proportion of correctly classified cases among all negative cases, measuring a recognition capability

$$spencificity = \frac{TN}{( FP + TN )}$$

of the classifier for negative cases. The balanced F-score is defined as a harmonic mean

$${F}_{1} = \frac{2}{\frac{1}{precision} + \frac{1}{recall}} = 2 \times \frac{precition \times recall}{recision + recall}$$

of accuracy and recall. Recall represents a proportion

$$recal = \frac{TP}{TP + FN}$$

of positive cases that the model can correctly predict among all actual positive cases. Sensitivity indicates a proportion of correctly classified cases among all positive cases, measuring a recognition capability

$$spencificity = \frac{TN}{( FP + TN )}$$

of the classifier for positive cases. AUC equals an area

$$AUC = \frac{\sum I ( {P}_{positive sample} , {P}_{negative sample} )}{M * N}$$

under the ROC curve, where

$$I ( {P}_{positive sample} , {P}_{negative sample} ) = \{ \begin{matrix}1 , & {P}_{positive sample} > {P}_{negative sample} \\ 0.5 , & {P}_{positive sample} = {P}_{negative sample} \\ 0 , & {P}_{positive sample} < {P}_{negative sample}\end{matrix} .$$

TABLE 2

| | top1 Acc | Specificity | F1 | Recall | Sensitivity | AUC |
|---|---|---|---|---|---|---|
| train | 99.43% | | | | | |
| test | 98.85% | 0.992 | 0.99 | 0.987 | 0.987 | 0.999 |

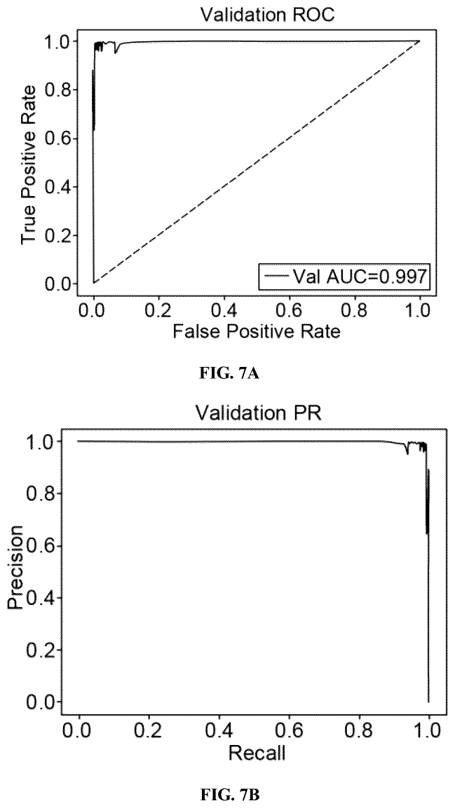

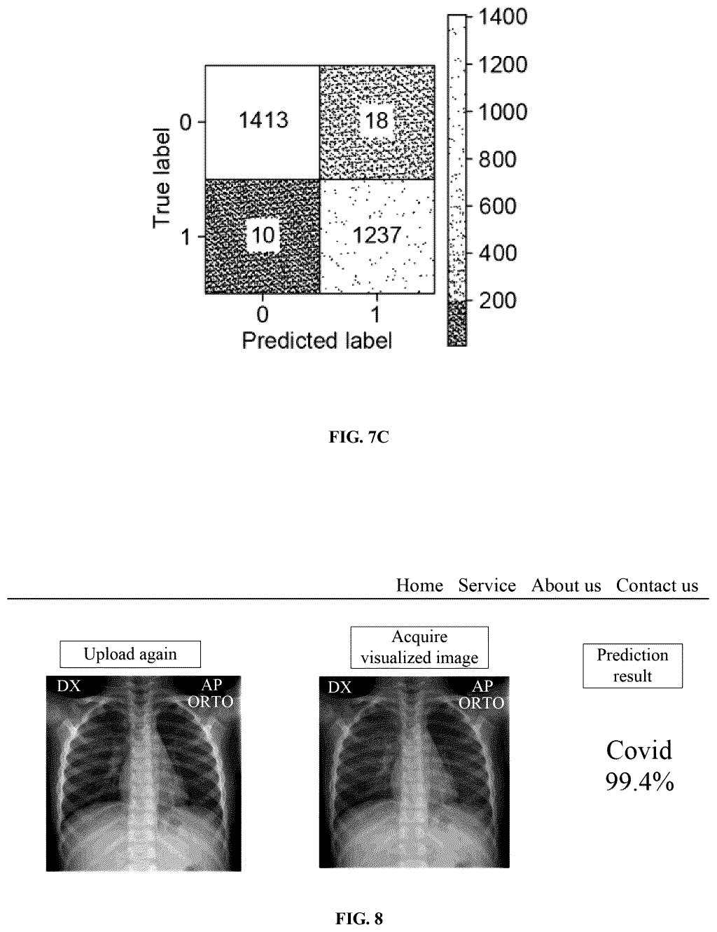

The changes in accuracy and loss during the training process are shown in FIG. 6, while the testing effect, including the ROC curve, PR curve, and confusion matrix, are illustrated in FIGS. 7A-7C. The final effect of the instance is shown in FIG. 8, specifically demonstrating the detection performance for COVID-19 chest X-rays. The middle image is an image after feature visualization. The graph on the right side displays the predicted categories and prediction probabilities. It can be seen that the present disclosure can accurately perform image classification tasks.

Each embodiment in the description is described in a progressive mode, each embodiment focuses on differences from other embodiments, and references can be made to each other for the same and similar parts between embodiments.

Specific examples are used herein for illustration of the principles and embodiments of the present disclosure. The description of the foregoing embodiments is used to help understand the method of the present disclosure and the core principles thereof. In addition, those of ordinary skill in the art can make various modifications in terms of specific embodiments and scope of application in accordance with the teachings of the present disclosure. In conclusion, the content of the description shall not be construed as limitations to the present disclosure.

1. A medical image auxiliary detection method using a Convolutional Block Attention Module (CBAM) mechanism-based residual network, comprising the following steps:
S1: acquiring a medical image, and clipping and normalizing the medical image;
S2: performing data transformation on the normalized medical image;
S3: establishing a network model on the basis of convolutional autoencoding, a feature extraction method using a spatial-and-channel attention mechanism, and a Hierarchical-Split (HS)-block module; and
S4: inputting the medical image obtained after the data transformation into the network model for prediction, and visualizing a predicted lesion region.

2. The medical image auxiliary detection method using a CBAM mechanism-based residual network according to claim 1, wherein step S1 specifically comprises the following steps:
S11: directly scaling the medical image to an image with a size suitable for input to the network model;
S12: converting the image into a grayscale image through channel reduction;
S13: converting the grayscale image into a tensor form (B, C, H, W), wherein B represents a batch size, C represents the number of image channels, H represents an image height, and W represents an image width; and
S14: normalizing the image obtained in S13 using a Normalize function.

3. The medical image auxiliary detection method using a CBAM mechanism-based residual network according to claim 1, wherein step S2 specifically comprises the following steps:
S21: performing data augmentation through central rotation of the normalized medical image to increase the amount of training data; and
S22: removing Gaussian noise from the normalized medical image using Gaussian filtering.

4. The medical image auxiliary detection method using a CBAM mechanism-based residual network according to claim 1, wherein step S3 specifically comprises the following steps:
S31: constructing a Res2Net residual network structure based on a ResNet network architecture;
S32: constructing a CBAM attention mechanism by combining a channel attention mechanism and a spatial attention mechanism, and inserting the constructed CBAM attention mechanism into the Res2Net residual network structure; and
S33: constructing an HS-block multi-level separable module and adding the HS-block multi-level separable module to a head of an entire network.

5. The medical image auxiliary detection method using a CBAM mechanism-based residual network according to claim 4, wherein a process of the channel attention mechanism is described as follows:
global average pooling and global maximum pooling are performed based on a width and a height of a network feature map; channel attention weights are obtained through a multi-layer perceptron; the obtained weights are summed element-wise;
finally, the weights are normalized using a Sigmoid function, and are then multiplied channel-wise to the original feature map.

6. The medical image auxiliary detection method using a CBAM mechanism-based residual network according to claim 4, wherein a process of the spatial attention mechanism is described as follows:
with input from the channel attention mechanism, global maximum pooling and global average pooling are performed on the feature map based on channels; then, the dimensionality is reduced to 1D through convolution operations, and attention features are generated through a Sigmoid function.

7. The medical image auxiliary detection method using a CBAM mechanism-based residual network according to claim 4, wherein said inserting the constructed CBAM attention mechanism into the Res2Net residual network structure specifically comprises:
inserting the constructed CBAM attention mechanism into a last layer of each residual block of ResNet.

8. The medical image auxiliary detection method using a CBAM mechanism-based residual network according to claim 4, wherein said constructing the HS-block multi-level separable module specifically comprises:
dividing the feature map into groups by channels, and performing cross-combination and convolution on different groups.

9. The medical image auxiliary detection method using a CBAM mechanism-based residual network according to claim 1, wherein step S4 specifically comprises:
extracting features from the model based on a Grad-CAM++ algorithm, plotting a heatmap, and overlaying the heatmap on an original image with 0.3 opacity.

10. The medical image auxiliary detection method using a CBAM mechanism-based residual network according to claim 9, wherein the Grad-CAM++ algorithm is specifically as follows:
a score for a specific class in a feature map is derived from a dot product of weights and the feature map, with a formula as follows:
Y^{c}=Σₖwₖ^{c}·ΣᵢΣⱼA_{i,j}^{k}; a corresponding heatmap formula is as follows:
L_{i,j}^{c}=Σₖwₖ^{c}·A_{i,j}^{k}, where A_{i,j}^{c}; calculation of weights uses gradients and a ReLU activation function for improvement, with a formula as follows:
$${w}_{k}^{c} = {\sum }_{i} {\sum }_{j} {∝}_{ij}^{kc} \cdot relu ( \frac{\partial {Y}^{c}}{\partial {A}_{ij}^{k}} ) .$$
